(19) United States
(12) Patent Application Publication
(10) Pub. No.: US 2021/0290835 A1
(43) Pub. Date: Sep. 23, 2021

(54) PLASMA BANKING TO MITIGATE AGING

(71) Applicant: Jay Yadav, Sandy Springs, GA (US)

(72) Inventor: Jay Yadav, Sandy Springs, GA (US)

(21) Appl. No.: 16/323,382

(22) PCT Filed: Aug. 4, 2017
(86) PCT No.: PCT/US2017/045469

§ 371 Date: Feb. 5, 2019

(51) Int. Cl. A61M 1/38; G16H 20/17; G16H 10/60; G16H 40/40; A61M 1/02

(52) U.S. Cl. CPC A61M 1/38; G16H 20/17; A61M 2202/0415; G16H 40/40; A61M 1/0272; G16H 10/60

(57) ABSTRACT

Disclosed herein are systems and methods that involve banking a subject's plasma so that it can be used later to mitigate the effects of aging.

### CROSS REFERENCE TO RELATED APPLICATION

This application claims priority to U.S. Provisional Patent Application No. 62/371,570 entitled “PLASMA BANKING TO MITIGATE AGING,” filed Aug. 5, 2016, the content of which is herein incorporated by reference in its entirety.

### BACKGROUND

Blood plasma transfusions from young subjects has been shown to restore mental capabilities in older subjects. However, there are ethical and safety issues in obtaining and using plasma from young donors. In addition to possible safety and efficacy risks inherent with allogenic transfusion, it also raises ethical concerns regarding exploitation of vulnerable populations.

### SUMMARY

Disclosed herein are systems and methods for banking a subject's plasma while they are young so that it can be used later as an autologous transfusion to mitigate aging. These systems and methods use a patient's own plasma instead of an unrelated donor, thereby removing the safety, efficacy, and ethical risks. The disclosed plasma bank therefore stores plasma samples from young subjects and catalogues these samples for later retrieval and auto-transfusion.

Therefore, disclosed is a method for mitigating aging of a subject comprising, collecting a plasma sample from a donor subject storing the plasma sample in a freezer under conditions suitable for autologous transplantation of the plasma back into the subject after a period of at least 10, 20, 30, 40, 50, or 60 years.

Also disclosed is a method for mitigating aging of a subject comprising transfusing the subject with an effective amount of a plasma sample that was collected from the subject and stored at least 5, 10, or 15 years prior to the transfusion.

In some embodiments, plasma collection occurs while the subject is at least 1 year age, but less than 25, 24, 23, 22, 21, 20, 18, 17, 16, 15, 14, 13, 12, 11, 10, 9, 8, 7, 6, 5, 4, 3, or 2 years of age. In some cases, the collection occurs once, collecting at least 0.1, 0.2, 0.3, 0.4, 0.5, 1.0, 1.5, or 2.0 liters of plasma. In other embodiments, the plasma is collected over at least 2, 3, 4, 5, 6, 7, 8, 9, or 10 donations, which can involve a plurality of ages.

Once collected, the plasma sample is stored in a freezer under conditions suitable to preserve the sample for at least 10, 15, 20, 25, 30, 35, 40, 45, 50, 55, 60, 65, 70 or more years. For example, the sample can be stored at a temperature less than −20° C., −30° C., −40° C., −50° C., −60° C., −80° C., −90° C., −100° C., −110° C., or −120° C. In some cases, the plasma samples are freeze dried (lyophilized) prior to storage.

Once the subject has advanced in age, the sample can be thawed and transfused back into the subject to mitigate the effects of aging. In some cases, the subject receives one transfusion. In some cases, the subject receives a plurality of transfusions. For example, the subject can receive transfusions every week, month, year, or decade, depending on the amount of sample collected and the age of the subject at the first transfusion. In some cases, the rate of transfusions is accelerated as the subject gets older, e.g., going from every decade, to every five years, to every year. An example transfusion schedule might be a transfusion at age 40, 50, 60, 65, 70, 75, 80, 81, 82, 83, 84, 85 years of age. In other embodiments, the subject receives a transfusion in response to specific signs of aging, such as loss of memory. In some of these embodiments, the method involves monitoring these signs and scheduling transfusions accordingly.

In other embodiments, the method involves concentrating one or more anti-aging factors from the plasma sample and administering those factors to the subject. In some embodiments, the method further involves treating the plasma, or agents isolated therefrom, with antibodies such as exogenous anti-aging factors.

Therefore, disclosed is a method for mitigating aging of a subject that involves collecting a plasma sample from the subject, freezing and storing the sample for at least 10, 15, 20, 25, 30, 35, 40, 45, 50, 55, 60, 65, 70, or more years, and then transfusing the sample back into the subject. Therefore, also disclosed is a method for mitigating aging of a subject that involves transfusing a plasma sample into the subject that had been collected from the subject and frozen for at least 10, 15, 20, 25, 30, 35, 40, 45, 50, 55, 60, 65, 70 or more years.

Also disclosed is a method that involves collecting a plasma sample from a donor subject as disclosed herein, freezing and storing the sample as disclosed herein, and cataloguing the sample according to the identity of the subject, date of collection, and location of sample in storage, in a manner suitable for retrieval of that sample for transfusion back into the subject. The method can further involve providing the donor subject with authentication information for retrieval of the sample from storage for transfusion, such as an account username and password. The method can also involve tracking the donor subject by age. For example, notices can be sent to the donor subject as they advance in age, or to the donor subject's physician, letting them know the volume and other parameters of their plasma samples. This can further include a proposed schedule for transfusion based on available volumes and collection dates.

One advantage of the disclosed methods is the ability of an older subject to get a transfusion of autologous young plasma. However, in some embodiments, the stored sample is used by a different subject. For example, the subject can be a relative or other allogenic subject. In these embodiments, the methods can further involve typing the sample for histocompatibility prior to transfusion. This information can also be included in the catalogue for retrieval based on histocompatibility.

Also disclosed is a plasma bank that contains a plurality of frozen plasma samples individually disposed within storage containers for use in mitigating aging. In some embodiments, the bank comprises plasma samples originating from at least 10, 50, 100, or 1000 different donor subjects. In some cases, a plurality of the samples were collected from donor subjects less than 25, 24, 23, 22, 21, 20, 18, 17, 16, 15, 14, 13, 12, 11, 10, 9, 8, 7, 6, 5, 4, 3, 2, or 1 years of age at the collection date. In some cases, the bank comprises a total of at least 0.1, 0.2, 0.3, 0.4, 0.5, 1.0, 1.5, or 2.0 liters of plasma for each at least 10, 50, 100, or 1000 of the donor subjects. In some embodiments, each plasma sample has a volume of at least 0.1, 0.2, 0.3, 0.4, 0.5, 1.0, 1.5, or 2.0 liters of plasma. In other embodiments, the samples can be pooled to reach these amounts.

In some embodiments, the samples of the plasma bank are stored in a freezer under conditions suitable to preserve the sample for at least 10, 15, 20, 25, 30, 35, 40, 45, 50, 55, 60, 65, 70, or more years. For example, the samples can be stored at a temperature about −20° C., −30° C., −40° C., −50° C., −60° C., −80° C., −90° C., −100° C., −110° C., or −120° C. Therefore, the plasma bank can also include at least one freezer, wherein the storage containers are disposed within the at least one freezer.

Another advantage of the disclosed plasma bank is the ability to retrieve a sample at the request of the donor subject for transfusion back into the donor subject. Therefore, in some cases plasma bank further comprises a catalogue comprising donor identity, collection date, and storage location for each serum sample. In addition, the catalogue can contain authentication information for each donor subject. In some cases, the catalogue further contains histocompatibility information.

In some cases, the catalogue comprises at least one database computer unit comprising at least one processing module and at least one memory device into which donor identity and collection date for each plasma sample is inputted, and at least one program code module that causes donor identity and collection date for each plasma sample to be displayed onto a display which is in communication with the database computer.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram depicting an embodiment cataloguing system.

### DETAILED DESCRIPTION

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

As used herein, the term “plasma” is intended to mean the fluid component of blood derived or obtained from a living organism (in dried or liquid form), prenatal, postnatal, mature or adult. The plasma may contain at least one clotting factor (in dried or liquid form) or component involved in clotting (in dried or liquid form). The term “clotting factor(s) or component(s) involved in clotting” is intended to include any blood component involved in clotting and includes, but is not limited to, Factor I, Factor II, Factor III, Factor V, Factor VI, Factor VII, Factor VIII, Factor IX, Factor X, Factor XI, Factor XII, Factor XIII, von Willebrands factor, Factor Ia, Factor Ia, Factor Ila, Factor Va, Factor VIa, Factor VIIa, Factor VIIIa, Factor IXa, Factor Xa, Factor Xla, Factor XIIa and Factor Xiiia, as well as components such as Protein C, Protein S, Serum or plasma Factor Pathway-Inhibitor (TFPI), Serum or plasma Factor (serum or plasma thromboplastin), procoagulant phospholipid, and thrombomodulin, all of which may be obtained or derived from any animal, including genetic, as well as chemically and biotechnically produced, variants thereof. The plasma according to the methods of the present invention may contain additives known in the art. Moreover, the plasma may be treated by methods known in the art, for instance by heating to reduce complement activity.

“Plasmapheresis” is a process in which plasma is separated and removed from blood cells of an individual using, for example, a cell separator. The separator may comprise a centrifuge and/or membrane with a pore size suitable to separate blood cells from plasma. The blood cells are returned to the individual and the plasma is collected and replaced within the donor's body with other fluids, by the normal compensatory mechanisms in such situations. Medication to keep the blood from clotting (an anticoagulant) may be mixed with the donor blood at the venipuncture site during the plasmapheresis.

Sample Collection

The disclosed methods involve collecting a plasma sample from the subject while they are young. In some embodiments, this collection occurs while the subject is less than 25, 24, 23, 22, 21, 20, 18, 17, 16, 15, 14, 13, 12, 11, 10, 9, 8, 7, 6, 5, 4, 3, 2, or 1 years of age. In some cases, the collection occurs once, collecting at least 0.1, 0.2, 0.3, 0.4, 0.5, 1.0, 1.5, or 2.0 liters of plasma. In other embodiments, the plasma is collected over at least 2, 3, 4, 5, 6, 7, 8, 9, or 10 donations, which can involve a plurality of ages. These collections are preferably stored separately, but can also be pooled to increase the total collected volume.

Plasma is produced when whole blood is collected in tubes that are treated with an anticoagulant. Whole blood can be collected into anticoagulant-treated tubes, e.g. EDTA-treated (lavender tops) or citrate-treated (light blue tops). Heparinized tubes (green tops) are indicated for some applications; however, heparin can often be contaminated with endotoxin, which can stimulate white blood cells to release cytokines. Cells can then be removed from plasma by centrifugation for 10 minutes at 1,000-2,000×g using a refrigerated centrifuge. Centrifugation for 15 minutes at 2,000×g depletes platelets in the plasma sample. The resulting supernatant is designated plasma. Following centrifugation, the liquid component (plasma) can be transferred into a clean polypropylene tube, e.g. using a Pasteur pipette.

In some cases, plasma is collected by plasmapheresis. During plasmapheresis, blood is initially taken out of the body through a needle or previously implanted catheter. Plasma is then removed from the blood by a cell separator. Three procedures are commonly used to separate the plasma from the blood cells, with each method having its own advantages and disadvantages. In discontinuous flow centrifugation, one venous catheter line is required. Typically, a 300 ml batch of blood is removed at a time and centrifuged to separate plasma from blood cells. In continuous flow centrifugation, two venous lines are used. This method requires slightly less blood volume out of the body at any one time, as it is able to continuously spin out plasma. In plasma filtration, two venous lines are used. The plasma is filtered using standard hemodialysis equipment. This continuous process requires that less than 100 ml of blood be outside the body at one time. After plasma separation, the blood cells are returned to the subject along with a plasma substitute.

Storage

The plasma sample is then stored in a freezer under conditions suitable to preserve the sample for at least 10, 15, 20, 25, 30, 35, 40, 45, 50, 55, 60, 65, 70 or more years. For example, the sample can be stored at a temperature of about −10° C., −15° C., −20° C., −30° C., −40° C., −50° C., −60° C., −80° C., −90° C., −100° C., −110° C., or −120° C.

Plasma should be frozen as rapidly as possible to avoid prolonged exposure of serum nutrients to higher salt concentrations. Water as the first serum component to freeze will become less dense, resulting in other serum components such as proteins and salts to accumulate at the bottom of the container at a higher concentration. Therefore, slow freezing can result in excessive formation of crystalline precipitates.

Multiple freeze/thaw cycles should be avoided as this could lessen the quality of the serum or plasma. Once the plasma has thawed, it should be stored refrigerated at 2° C. to 8° C. for no longer than 30 days. Therefore, in preferred embodiments, the plasma is aliquoted into unit amounts appropriate for a single transfusion. In some embodiments, smaller aliquots are also made for periodic evaluation. Therefore, the serum can be aliquoted into volumes that include from about 0.1 mL to 1000 mL, such as 0.1, 0.5, 1, 5, 10, 50, 100, 200, 250, 500, 1,000, or 2,000 mL.

In some cases, the plasma samples are freeze dried (lyophilized). Lyophilization works by freezing the material and then reducing the surrounding pressure to allow the frozen water in the material to sublimate directly from the solid phase to the gas phase.

In some embodiments, the method involves concentrating one or more anti-aging factors from the blood, serum, or plasma sample and storing those factors.

In some embodiments, the plasma samples are banked, which involves cataloguing the sample for future use. One advantage of the disclosed plasma bank is the ability to retrieve the sample at the request of the donor subject for transfusion back into the donor subject. Therefore, in some cases, the disclosed plasma bank further comprises a catalogue comprising donor identity, collection date, and storage location for each plasma sample. In addition, the catalogue can contain authentication information for each donor subject. In some cases, the catalogue further contains histocompatibility information.

The disclosed bank can therefore contain a plurality of frozen plasma samples individually disposed within storage containers for use in mitigating aging. In some embodiments, the plasma bank can also include at least one freezer, wherein the storage containers are disposed within the at least one freezer.

In some embodiments, the bank comprises plasma samples originating from at least 10, 50, 100, or 1000 different donor subjects. In some cases, a plurality of the samples were collected from donor subjects less than 12, 13, 14, 15, 16, 17, or 18 years of age at the collection date.

In some cases, the plasma bank comprises a total of at least 0.1, 0.2, 0.3, 0.4, 0.5, 1.0, 1.5, or 2.0 liters of plasma for each at least 10, 50, 100, or 1000 of the donor subjects. In some embodiments, each plasma sample has a volume of at least 0.1, 0.5, 1, 5, 10, 50, 100, 200, 250, 500, 1,000, or 2,000 mL of plasma. In some embodiments, each serum sample has a volume of about one pint of plasma. In other embodiments, the samples can be pooled to reach these amounts.

In some embodiments, the method further involves tracking the donor subject by age.

In some cases, the catalogue comprises at least one database computer unit comprising at least one processing module and at least one memory device into which donor identity, collection date, and sample location in storage for each serum sample is inputted, and at least one program code module that causes donor identity, collection date, and sample location for each plasma sample to be displayed onto a display which is in communication with the database computer.

In one embodiment, the catalogue may comprise a database (130) stored on a non-volatile computer readable memory hardware component (102) connected to at least one processor (101), such that the processor is responsive to a set of computerized instructions also stored in the memory as executable software (104).

In many embodiments, the database will be implemented on a server (50) and accessible by any one of several client computing devices (185). The client computing devices are in data communication with the server such that the client computing devices are configured to issue database manipulation commands to the server, typically across a communications network (175). A server communications interface (150) receives the client's database manipulation commands to implement a database manipulation process. The database manipulation process may include, but is not limited to, searching the database according to the client commands, extracting or copying data content from the database for transmission back to the client computing devices, or displaying data content from the database on an output device (195) (e.g., a screen or a monitor) at the server or on the client computing device.

In one embodiment, the data content of interest to a user of the server (50) or client device (185) may include the above-referenced information identifying objective parameters related to a given sample tracked in the database (130), such as, but not limited to, identity of the subject donating the sample, a date of sample collection, a physical location related to the storage location for the sample, the donor's authentication information proving identity and security credentials necessary for accessing a sample, and any other information necessary to document objective parameters pairing a particular sample with a respective subject donor or other authorized individual allowed to gain access to or possession of any respective sample tracked in the database.

The above-noted database may be configured to accommodate the fact that samples acquired, tracked, and stored according to a plasma banking system will be used for transfusion back into a donor subject or into another authorized individual many years, if not decades after collection. Many of the samples tracked for a single subject donor may be acquired across numerous and diverse geographic locations and across spans of time that vary for each subject donor. Accordingly, the plasma banking system accommodates storing the data content described above across databases stored in separate locations and on separate computerized hardware components. For example, a single subject donor may deposit multiple samples, and each respective sample may have a corresponding database entry in multiple respective tables that correspond to at least one database stored in separate memory components (e.g., 102A, 102B, 102C, and so on) of separate servers (50A, 50B, 50C, 50D) (i.e., the plasma banking system accommodates using cloud based storage servers with distributed memory components).

The cataloguing described above includes particular software based features (104), implemented by appropriate computer hardware such as a memory (102) and a processor (101), to address notable facets of the plasma banking system that arise in particular because of several inherent characteristics including, but not limited to:

(i) the large spans of time between sample donation and sample use;

(ii) the geographic differences inherent in collecting, storing, and distributing the samples;

(iii) the varying donation habits of subject donors.

In regard to the first item, the cataloguing system described herein accommodates expected changes in the kinds of data that a database storing the above described information may need to account for to be relevant over the decades. In one embodiment, the cataloguing step of a method, implemented as a plasma banking system, may include the step of accumulating and storing metadata (120) about the data content of the database. This metadata (120) is intended to describe each parameter stored in the database in a convenient, descriptive, and possibly layman's vocabulary. Over the course of the life of the database, the metadata may be updated to relate respective pieces of data content with more modern, timely, or relevant terminology that is quickly searched, shared or transmitted between appropriate computer components on a network. The metadata may be encompassed within the database itself, within a separate file or memory location in the non-volatile computerized memory, or in another location all together so long as the metadata is conveniently placed for updating and searching archived and new data points regarding samples collected for the plasma banking system. In some embodiments, the metadata may be transmitted back and forth across the network (150) as necessary.

The problem of geographic differences among sample collection, storage, and distribution points presents numerous opportunities for the plasma banking system to remain relevant over time, particularly the long periods of time relevant to the system described herein. The individual samples may be physically marked, labeled, or identified with geographic locations for each donation. In fact, so long as the above noted database is annotated appropriately, moving the discrete samples over time to locations in proximity to either the subject donor or another authorized individual approaching an age to begin transfusions is entirely feasible and helpful. In this regard, the cataloguing features described in this disclosure also serve the purpose of a logistics control system based upon sample parameters such as an age of a sample, an age of a subject donor or other authorized recipient of the sample, and the geographic locations of samples, donors, and other recipients at any point in time. The logistics control system allows for not only gathering the samples across varying geographies and time periods, but also preparing the samples for convenient use in a proper geographic location adjusted as a subject donor reaches ages closer to using the samples.

As noted above, donors who provide samples at younger ages will develop patterns of donation habits as the donor's age, and those habits must be accounted for in the cataloging system in a useful way. The database described herein, therefore, anticipates that the samples, being tracked in the cataloguing features of the system, may have highly variable data entries and even null values for certain fields at different points in time with donor habits being entirely specific to each donor. In this regard, the cataloguing system may be characterized as dynamically adjusting data placement across separate tables or separate databases with appropriate structures for subject donor sample entries as a subject's donation pattern accrues over time. One non-limiting way in which the cataloguing component of the plasma banking system adjusts to entirely diverse donation patterns is by utilizing data placement algorithms to minimize null values in any given table so that donors with similar or compatible donation habits are stored in databases with similar structures for housing data content. In this way, the plasma banking system utilizes a database cataloguing system that is dynamically adjusted over time to gain artificial intelligence regarding subject donors and respective data points that become available at entirely random time intervals for each sample and each donor.

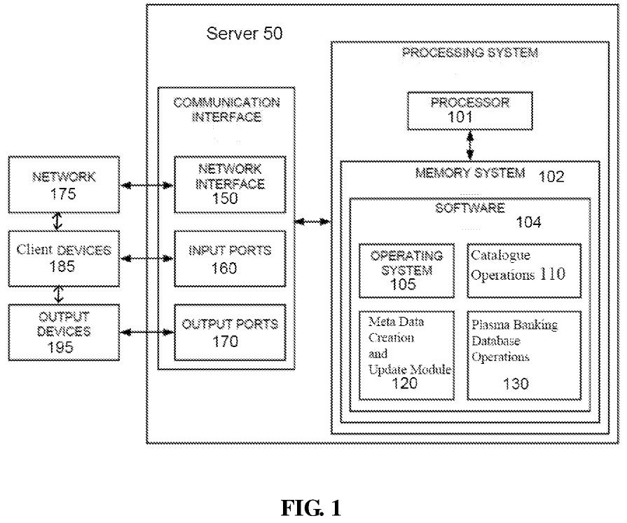

The cataloguing system according to this disclosure may be implemented according to FIG. 1, which is shown merely as an example, and, is in no way limiting of the system and method described herein.

Methods of Mitigating Aging with Autologous Serum

Once the subject has advanced in age, the sample can be thawed and transfused back into the subject to mitigate the effects of aging. In some cases, the subject receives one transfusion. In some cases, the subject receives a plurality of transfusions. For example, the subject can receive transfusions every week, month, year, or decade, depending on the amount of sample collected and the age of the subject at the first transfusion. In some cases, the rate of transfusions is accelerated as the subject gets older, e.g., going from every decade, to every five years, to every year. An example transfusion schedule might be a transfusion at age 40, 50, 60, 65, 70, 75, 80, 81, 82, 83, 84, 85 years of age. In other embodiments, the subject receives a transfusion in response to specific signs of aging, such as loss of memory. In some of these embodiments, the method involves monitoring these signs and scheduling transfusions accordingly.

In some cases, the subject receives one transfusion. In some cases, the subject receives a plurality of transfusions. For example, the subject can receive transfusions every week, month, year, or decade, depending on the amount of sample collected and the age of the subject when they receive the first transfusion. In some cases, the subject receives accelerates the rate of transfusions as they get older, e.g., going from every decade, to every five years, to every year. An example transfusion schedule might be a transfusion at age 40, 50, 60, 65, 70, 75, 80, 81, 82, 83, 84, 85 years of age. In other embodiments, the subject receives a transfusion in response to specific signs of aging, such as loss of memory. In some of these embodiments, the method involves monitoring these signs and scheduling transfusions accordingly.

Plasma can be transfused as fresh-frozen plasma (FFP) for 24 hours after thawing. It can then be stored at refrigerator temperature (1-6° C.) for an additional 4 days and transfused as “thawed plasma.”

In some cases, the thawed plasma is administered at a dose of about 5 to 30 mL/kg body weight, including about 10-15 mL/kg of body weight.

A number of embodiments of the invention have been described. Nevertheless, it will be understood that various modifications may be made without departing from the spirit and scope of the invention. Accordingly, other embodiments are within the scope of the following claims.

1. A method for mitigating aging of a subject comprising, collecting a plasma sample from a donor subject storing the plasma sample in a freezer under conditions suitable for autologous transplantation of the plasma back into the subject after a period of at least 10, 20, 30, 40, 50, or 60 years.

2. A method for mitigating aging of a subject comprising transfusing the subject with an effective amount of a plasma sample that was collected from the subject and stored at least 5, 10, or 15 years prior to the transfusion.

3. A plasma bank comprising a
(a) a plurality of frozen plasma samples individually disposed within storage containers, and wherein the bank comprises a total of at least 250, 500, or 1000 mL of plasma for at least 10, 50, 100, or 1000 of the donor subjects; and
(b) a catalogue comprising donor identity and collection date for each plasma sample.

4. The plasma bank of claim 3, wherein the bank comprises plasma samples originating from at least 10, 50, 100, or 1000 different donor subjects less than 16 years of age at the collection date.

5. The plasma bank of claim 3, wherein each plasma sample has a volume of at least 100 ml, 250 mL, 500 mL, or 1000 mL.

6. The plasma bank of claim 3, wherein the plasma samples are stored at a temperature less than −120° C.

7. The plasma bank of claim 3, wherein donor-specific plasma samples are retrievable at the request of the donor subject for transfusion.

8. The plasma bank of claim 7, wherein the catalogue comprises authentication information for each donor subject.

9. The plasma bank of claim 3, wherein the catalogue comprises at least one database computer unit comprising at least one processing module and at least one memory device into which donor identity and collection date for each serum sample is inputted, and at least one program code module that causes donor identity and collection date for each blood, serum, or plasma sample to be displayed onto a display which is in communication with the database computer.

10. The plasma bank of claim 3, further comprising at least one freezer, wherein the storage containers are disposed within the at least one freezer.

11. A method comprising
(a) collecting a plasma sample from a donor subject;
(b) storing the plasma sample in a freezer under conditions that can preserve plasma protein activity for at least 60 years; and
(c) cataloguing the sample according to the identity of the subject in a manner suitable for retrieval of that sample for transfusion back into the subject.

12. The method of claim 11, wherein the plasma samples are stored at a temperature less than −60° C.

13. The method of claim 12, further comprising providing the donor subject with authentication information for retrieval of the sample from storage for transfusion.

14. The method of claim 13, wherein the authentication information comprises an account username and password.

15. The method of claim 11, further comprising tracking the donor subject by age.
